(19) United States
(12) Patent Application Publication
(10) Pub. No.: US 2007/0255213 A1
(43) Pub. Date: Nov. 1, 2007

(54) Tube and method of producing the same

(75) Inventors: Akira Itoh, Kariya-city (JP); Masahiro Shimoya, Kariya-city (JP); Kazue Noguchi, Kariya-city (JP); Hiroyuki Genda, Kariya-city (JP)

Correspondence Address: HARNESS, DICKEY & PIERCE, P.L.C., P.O. BOX 828, BLOOMFIELD HILLS, MI, 48303, US

(73) Assignee: DENSO Corporation, Kariya-city (JP)

(21) Appl. No.: 11/788,981

(22) Filed: Apr. 23, 2007

(30) Foreign Application Priority Data
Apr. 26, 2006 (JP) 2006-121989

(51) Int. Cl. A61F 7/12

(52) U.S. Cl. 604/113

(57) ABSTRACT

A tube for exchanging heat between inside fluid and outside fluid includes a uniform cross-section portion. The outside fluid flows in an outside passage meanderingly extending in an outside fluid flowing direction. The uniform cross-section portion is disposed at least at each end of the tube longitudinal direction, and has an inside passage and a joint. The inside passage is constructed with a first inner face and a second inner face, which are spaced from each other, and the inside fluid flows through the inside passage. The joint is constructed with the first inner face and the second inner face, which are connected to each other to increase a pressure-resistant strength.

### CROSS REFERENCE TO RELATED APPLICATION

This application is based on Japanese Patent Application No. 2006-121989 filed on Apr. 26, 2006, the disclosure of which is incorporated herein by reference in its entirety.

### BACKGROUND OF THE INVENTION

1. Field of the Invention

The present invention relates to a tube having an outside fluid passage extending in an outside fluid flowing direction.

2. Description of Related Art

U.S. Pat. No. 6,595,273 (corresponding to JP-A-2004-3787) discloses a tube constructed with two board members. Each of the board members has a base and plural protrusions protruding from the base. The two board members are faced to each other such that the protrusions of the board members face to each outside. An overlap part is set between the protrusions of the board members, and works as an inside fluid passage, through which inside fluid flows.

The protrusion meanderingly extends in an outside fluid flowing direction. Plural recesses (outside fluid passages) are provided between adjacent protrusions, and meanderingly extends in the outside fluid flowing direction. Therefore, outside fluid flowing adjacent to an outer wall of the tube flows along the recesses meanderingly, so that a flow of the outside fluid is agitated. Thereby, a thermal boundary layer generated adjacent to the outer wall of the tube can be made thinner, so that heat-transmitting rate can be improved.

Further, the board members are connected to each other such that bottom parts of the recesses of the board members are in contact with each other. Therefore, the recess can enhance pressure-resistant strength of the tube as an inner pillar.

Here, a heat exchanger is constructed with a plurality of the tubes and tanks disposed at ends of the tube, respectively. The tank distributes inside fluid into the plural tubes, or collects inside fluid from the plural tubes. That is, the end of the tube is inserted into an insert hole of the tank, and connected to an edge of the insert hole by brazing. Thus, the tube and the tank can be connected.

However, cross-section shape of the tube in the tube longitudinal direction is not uniform, because the plural recesses having the meandering shape are formed on the tube. Therefore, a part of the edge of the insert hole of the tank may overlap with a part of the recess. In this case, a relatively large gap is formed between the edge of the insert hole of the tank and the recess, so that a defective connection (brazing) between the tube and the tank can be easily generated.

Here, the cross-section shape of the tube in the tube longitudinal direction can be uniform, if the end of the tube is made flat by eliminating the recess. However, because the recesses enhance the pressure-resistant strength of the tube, the pressure-resistant strength of the end of the tube may be reduced, if the end of the tube is made flat.

### SUMMARY OF THE INVENTION

In view of the foregoing and other problems, it is an object of the present invention to provide a tube having a predetermined pressure-resistant strength. Further, the tube can be easily and accurately connected to a tank.

According to a first example of the present invention, a tube for exchanging heat between inside fluid and outside fluid includes a uniform cross-section portion. Inside fluid flows in the tube in a tube longitudinal direction, and outside fluid flows in an outside passage meanderingly extending in an outside fluid flowing direction outside of the tube. The uniform cross-section portion is disposed at least at each end of the tube longitudinal direction, in which a cross-section is approximately uniform in the tube longitudinal direction. The uniform cross-section portion has an inside passage and a joint. The inside passage is constructed with a first inner face and a second inner face, which are spaced from each other, and inside fluid flows through the inside passage. The joint is constructed with the first inner face and the second inner face, which are connected to each other to increase a pressure-resistant strength. The joint and the first passage are alternately arranged in the uniform cross-section portion in a direction approximately perpendicular to the tube longitudinal direction.

According to a second example of the present invention, a tube for exchanging heat between a first fluid flowing inside of the tube and a second fluid flowing outside of the tube includes a uniform cross-section portion and an outside passage portion. The uniform cross-section portion, in which a cross-section is approximately uniform in a longitudinal direction, has an inside passage and a joint. The inside passage is constructed with a first inner face and a second inner face, which are spaced from each other, and the first fluid flows through the inside passage. The joint is constructed with the first inner face and the second inner face, which are connected to each other to increase a pressure-resistant strength. The joint and the inside passage are alternately arranged in the uniform cross-section portion in a width direction approximately perpendicular to the longitudinal direction. In the outside passage portion, an outside passage meanderingly extends in the width direction, and the second fluid flows through the outside passage. The uniform cross-section portion is disposed at least two ends of the outside passage portion in the longitudinal direction.

Accordingly, the tube can have a predetermined pressure-resistant strength, and can be easily and accurately connected to the tank.

### BRIEF DESCRIPTION OF THE DRAWINGS

The above and other objects, features and advantages of the present invention will become more apparent from the following detailed description made with reference to the accompanying drawings. In the drawings:

FIG. 1 is a schematic perspective view showing a heat exchanger according to an embodiment of the present invention;

FIG. 2 is a perspective view showing a tube of the heat exchanger;

FIG. 3 is a schematic plan view showing the tube;

FIG. 4 is a perspective view showing a heat-exchanging portion of the heat exchanger;

FIG. 5A is a perspective view showing a process of forming the tube, and FIG. 5B is a schematic perspective view showing a process of bending the tube; and

FIG. 6 is a perspective view showing another method of producing the tube.

### DETAILED DESCRIPTION OF PREFERRED EMBODIMENT

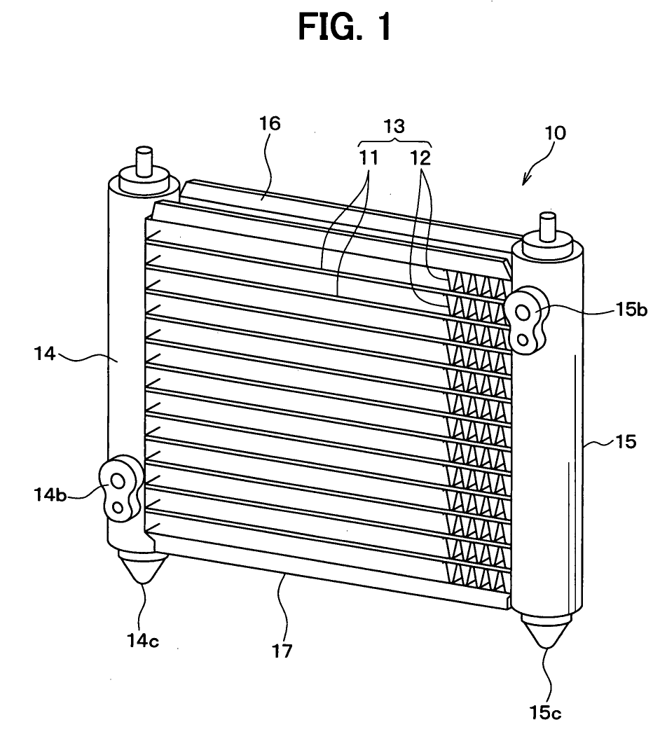

A heat exchanger 10 shown in FIG. 1 is typically used as a condenser for condensing refrigerant in a refrigeration cycle of a vehicle air-conditioning apparatus. The heat exchanger 10 is arranged at a position in an engine compartment of a vehicle, at which outside air is readily received when the vehicle is running.

In the heat exchanger 10, heat is exchanged between refrigerant (inside fluid) and air (outside fluid). When refrigerant is discharged from a compressor (not shown) of the refrigeration cycle, the refrigerant has high-temperature and high-pressure. The refrigerant can be condensed in the heat exchanger 10. Specifically, as shown in FIG. 1, the heat exchanger 10 includes a heat-exchanging portion 13, which is constructed with plural flat tubes 11 and plural corrugated fins 12. Refrigerant flows through a refrigerant passage (inside passage) in the tube 11. The heat exchanger 10 further includes tanks 14, 15 at ends of the heat-exchanging portion 13 in the tube longitudinal direction, respectively.

The tank 14, 15 distributes refrigerant into the tubes 11, or collects refrigerant from the tubes 11. Aside plate 16, 17 connects the tanks 14, 15 at ends of the tanks 14, 15 in the tank longitudinal direction. The side plate 16, 17 is approximately parallel to the tubes 11, and holds an outer shape of the heat exchanger 10 having a rectangular shape. The tubes 11, the fins 12, and the tanks 14, 15 are integrally connected by brazing.

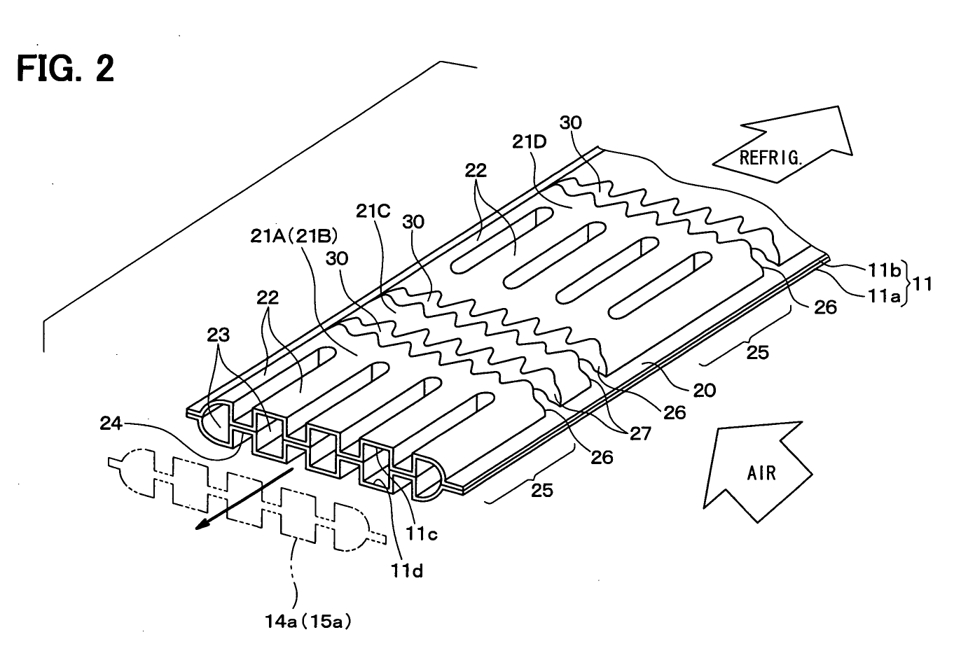

The tank 14, 15 is made of an aluminum-base material clad (covered) with a brazing material (filler), and has a cylinder shape. The tank 14, 15 has plural insert holes 14a, 15a in the tube width direction, as shown in FIG. 2. An end of the tube 11 is inserted into the insert hole 14a, 15a of the tank 14, 15.

A connection block 14b is brazed to a lower side of the tank 14 in the tank longitudinal direction. An inlet pipe (not shown) is to be connected to the connection block 14b in order to introduce the high-temperature and high-pressure refrigerant discharged from the compressor (not shown) of the refrigeration cycle into the tank 14. Further, an engaging protrusion 14c is fitted to a lower end of the tank 14 in the tank longitudinal direction. The heat exchanger 10 is mounted to the vehicle through the engaging protrusion 14c.

A connection block 15b is brazed to an upper side of the tank 15 in the tank longitudinal direction. An outlet pipe (not shown) is to be connected to the connection block 15b in order to discharge liquid-phase refrigerant into an expansion valve (not shown) of the refrigeration cycle from the tank 15. Further, an engaging protrusion 15c is fitted to a lower end of the tank 15 in the tank longitudinal direction. The heat exchanger 10 is mounted to the vehicle through the engaging protrusion 15c.

FIG. 2 shows an end of the tube 11 to be connected to the tank 14. The other end of the tube 11 to be connected to the tank 15 is similar to FIG. 2. The tube 11 is constructed with two board members 11a, 11b. The board member 11a, 11b has a flat base 20 and emboss parts 21A, 21B, 21C, 21D protruding from the flat base 20. The board member 11a, 11b is made of a clad material, which is an aluminum-base thin board covered with a brazing material. The board members 11a, 11 b oppose to each other such that the emboss parts 21A-21D face each outside. The refrigerant passage is constructed between the emboss part 21A-21D of the board member 11a and the emboss part 21A-21D of the board member 11b. Thus, refrigerant can flow between the board members 11a, 11 b.

The emboss part 21A-21D protrudes from the base 20 at an approximately center part of the board member 11a, 11b in the narrow side direction (air-flowing direction). The protruding top face of the emboss part 21A-21D is flat. The emboss part 21A is arranged at an end portion of the board member 11a, 11b in the longitudinal direction, and to be connected to the tank 14. The emboss part 21B is arranged at the other end portion of the board member 11a, 11b in the longitudinal direction, and to be connected to the tank 15. The emboss part 21A, 21B adjacent to the end of the tube 11 has plural comb-teeth 22 extending in the tube longitudinal direction.

The comb-teeth 22 of the emboss part 21A, 21B has a linear shape extending in the tube longitudinal direction. Because the comb-teeth 22 of the board member 11a and the comb-teeth 22 of the board member 11b overlap with each other, a refrigerant passage 23 (inside passage) is constructed between an inner face 11c of the board member 11b and an inner face 11d of the board member 11a. The inner face 11c and the inner face 11d oppose to each other, and are spaced from each other.

A plurality of the refrigerant passages 23 is arranged in a direction perpendicular to the tube longitudinal direction. Further, the base 20 between adjacent refrigerant passages 23 works as a joint 24 for increasing pressure-resistant strength, because the inner face 11c and the inner face 11d are connected to each other at the joint 24.

The refrigerant passage 23 and the joint 24 are alternately arranged in a direction perpendicular to the tube longitudinal direction. The board member 11a, 11b has a uniform cross-section portion 25 at both end portions of the board member 11a, 11b in the tube longitudinal direction. The uniform cross-section portion 25 of the tube 11 has a uniform cross-section in the tube longitudinal direction.

A double-chained line of FIG. 2 represents the insert hole 14a (15a) of the tank 14 (15). The insert hole 14a (15a) has a shape corresponding to the cross-section of the uniform cross-section portion 25 of the tube 11, so that a gap between the insert hole 14a (15a) and an outer face of the tube 11 can be reduced.

The emboss part 21A (21B) has a curved surface 26 meanderingly extending in the air-flowing direction, and the curved surface 26 opposes to the emboss part 21C. The emboss parts 21C, 21D are alternately arranged in a mid-part between the emboss parts 21A, 21B in the tube longitudinal direction. The emboss part 21C has a curved surface 27 meanderingly extending in the air-flowing direction at its each end in the tube longitudinal direction.

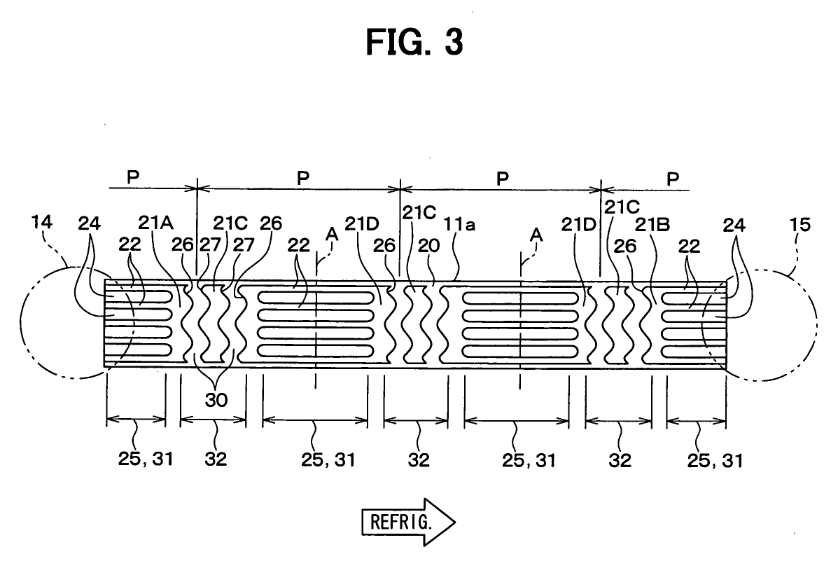

FIG. 3 shows a schematic plan view of the board member 11a, which is approximately similar to the board member 11b. When the emboss part 21D is separated into a left half and a right half by a chained line A in FIG. 3, a shape of the left half of the emboss part 21D is similar to a shape of the emboss part 21B, and a shape of the right half of the emboss part 21D is similar to a shape of the emboss part 21A. Therefore, the refrigerant passage 23 and the joint 24 can be constructed by the emboss part 21D and the base 20, so that the uniform cross-section portion 25 can be formed.

An air passage 30 (outside passage) meanderingly extending in the air-flowing direction is provided between the emboss parts 21A-21D, that is, between the curved surfaces 26, 27. The air passage 30 will be described below.

A shape pattern P constructed with the emboss part 21C and the emboss part 21D is repeatedly formed on the board member 11a, 11b in the tube longitudinal direction. Therefore, a first range 31 having the uniform cross-section portion 25 and a second range 32 having the air passage 30 are alternately arranged on the board member 11a, 11b in the tube longitudinal direction.

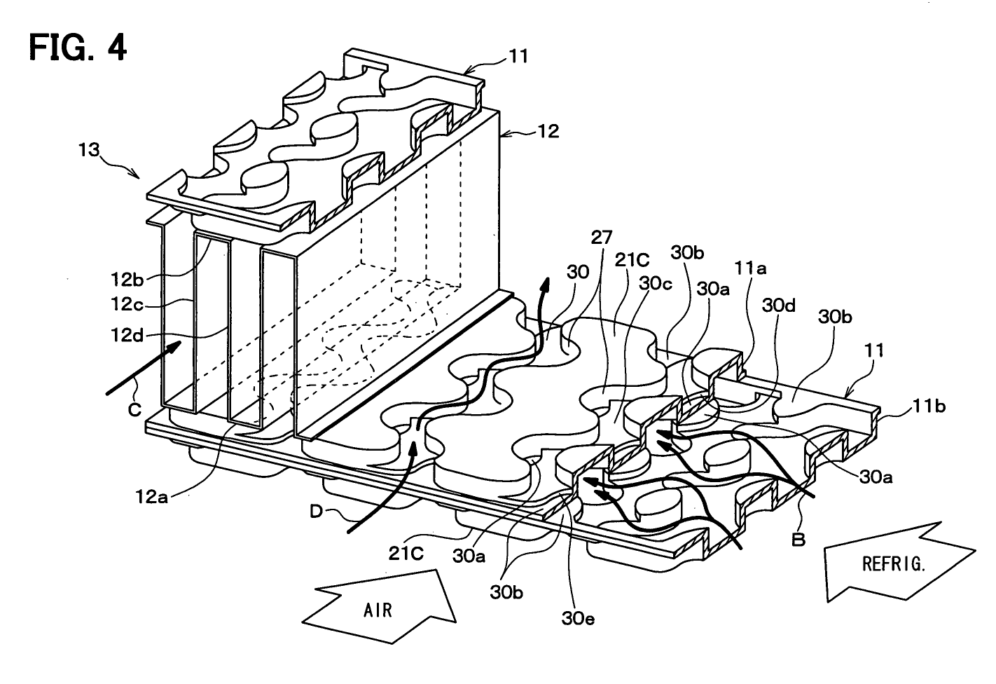

The air passage 30 will be described with reference to FIG. 4, in which plural emboss parts 21C are shown in place of the emboss parts 21A, 21B, 21D for convenience of the description. The air passage 30 is constructed with a top part 30a, an end part 30b and a flat face 30c. The top part 30a represents a top of the meandering shape, and the end part 30b represents an end portion of the air passage 30 in the air-flowing direction. The top part 30a and the end part 30b are constructed with the base 20. The flat face 30c is formed by slightly embossing the tube 11 outward.

Further, the air passage 30 of the board member 11a and the air passage 30 of the board member 11b are arranged offset relative to each other in the tube longitudinal direction. The air passage 30 of the board member 11a and the air passage 30 of the board member 11b overlap with each other at the top part 30a and the end part 30b.

Therefore, the top part 30a of the board member 11a and the top part 30a of the board member 11b are in contact with each other, and the end part 30b of the board member 11a and the end part 30b of the board member 11b are in contact with each other. Thus, the board members 11a, 11 b are connected to each other through the top parts 30a and the end parts 30b.

A step part 30d is provided between the top part 30a and the flat face 30c. A step part 30e is provided between the end part 30b and the flat face 30c. Each of the step parts 30d, 30e is set to have a height of 0.65 mm in this embodiment.

As shown of an arrow B in FIG. 4, the refrigerant passage 23 is arranged in the tube 11, and meanders complicatedly. Specifically, because the air passage 30 of the board member 11a and the air passage 30 of the board member 11b are arranged offset relative to each other in the tube longitudinal direction, the refrigerant passage 23 meanders in the tube height direction (up-and-down direction in FIG. 4), and extends in the tube longitudinal direction.

Further, because the board members 11a, 11b are connected to each other through the top parts 30a, the refrigerant passage 23 is branched at the top parts 30a. Then, the branched passages join together again at a downstream side of the top parts 30a. By repeating the branches and the joins, the refrigerant passage 23 meanders in the tube width direction parallel to the air-flowing direction, and extends in the tube longitudinal direction.

In addition, as shown in FIG. 4, the fin 12 is a corrugated fin, which is formed by bending a thin board made of a bare aluminum-base material not covered with a brazing material into a rectangular wave shape. The fin 12 has a connection part 12a, 12b to be connected to the emboss part 21A-21D, and the connection part 12a, 12b has a flat shape. The fin 12 further has a flat face 12c, 12d extending in the tube arrangement direction (up-and-down direction in FIG. 4). The flat face 12c, 12d has plural louvers (not shown) for opposing to air flow.

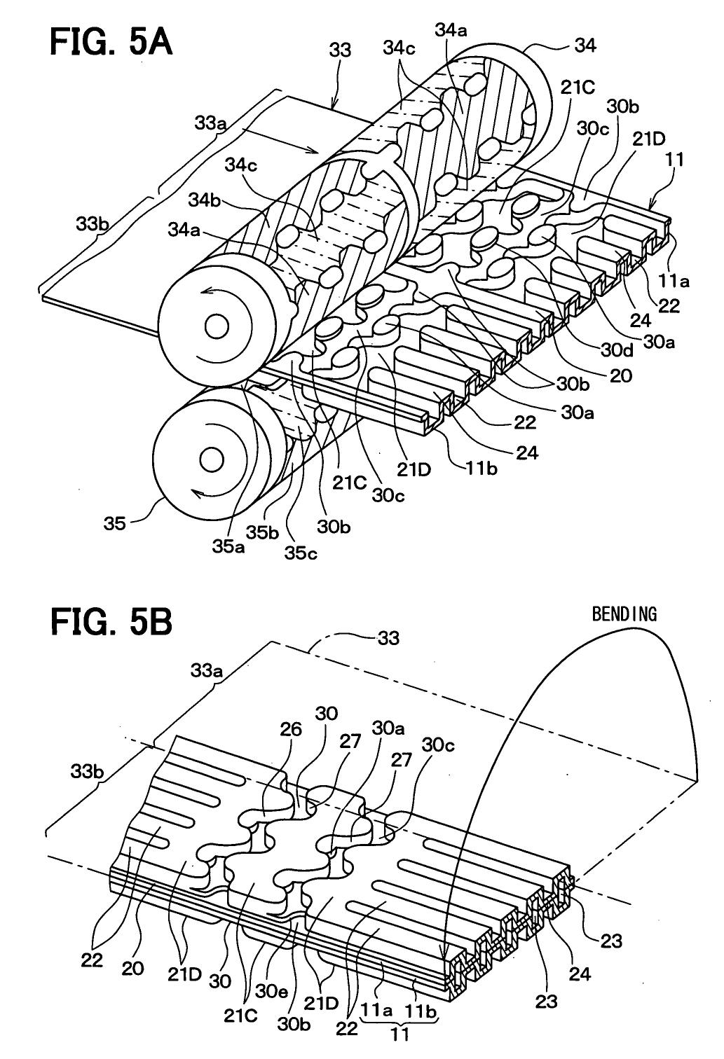

Next, a method of producing the tube 11 will be described. As shown in FIG. 5A, a work 33 having a band plate shape to be the base 20 of the tube 11 is pressed between rollers 34, 35. Thus, the emboss parts 21C, 21D and the flat face 30c of the air passage 30 are continuously embossed from the work 33.

The board members 11a, 11b are integrally formed by the single work 33. The work 33 has a first area 33a for the board member 11a and a second area 33b for the board member 11b in the narrow side direction. The emboss parts 21C, 21D and the flat face 30c of the air passage 30 are embossed from the first area 33a and the second area 33b at the same time.

The pair of the rollers 34, 35 embosses the emboss parts 21C, 21D and the flat face 30c corresponding to the shape pattern P. The roller 34 has a first protrusion 34a and a second protrusion 34b for embossing the emboss parts 21C, 21D. The roller 34 further has a third protrusion 34c for embossing the flat face 30c. The third protrusion 34c is smaller than the first and second protrusions 34a, 34b. The first, second and third protrusions 34a, 34b, 34c are provided in the circumferential direction. The roller 35 has first, second and third recesses 35a, 35b, 35c each corresponding to the first, second and third protrusions 34a, 34b, 34c of the roller 34 in the circumferential direction.

Therefore, when the rollers 34, 35 continuously revolve, the shape pattern P is repeatedly formed such that a longitudinal direction of the work 33 corresponds to the tube longitudinal direction. In addition, in FIG. 5A, the first and second protrusions 34a, 34b and the first and second recesses 35a, 35b have solid-line hatch, and the third protrusion 34c and the third recess 35c have chained-line hatch, for convenience.

Then, as show in FIG. 5B, the work 33 is bent at an approximately center line of the work 33 in the narrow side direction, and ends of the work 33 in the narrow side direction are made in contact with each other. In the bending process, a part of the ends of the work 33 in the narrow side direction are crimped in order to temporarily fix the work 33 in the bent state. (The crimping process is not shown in the drawings.)

Thereafter, the work 33 is cut such that a predetermined emboss part 21D is separated into the uniform cross-section areas 25 in the longitudinal direction. Thereby, the emboss parts 21A, 21B can be formed at the ends of the tube 11 in the tube longitudinal direction, respectively.

Then, the base 20 of the first area 33a and the base 20 of the second area 33b are connected by brazing, after the tubes 11, the fins 12, and the tanks 14, 15 are temporarily assembled. Thus, the tubes 11, the fins 12 and the tanks 14, 15 can be integrally brazed at the same time. However, the brazing may be performed relative to the single tube 11.

Further, the embossing may be performed by pressing with a forming die, although the embossing is performed by rolling with the rollers 34, 35, as described above.

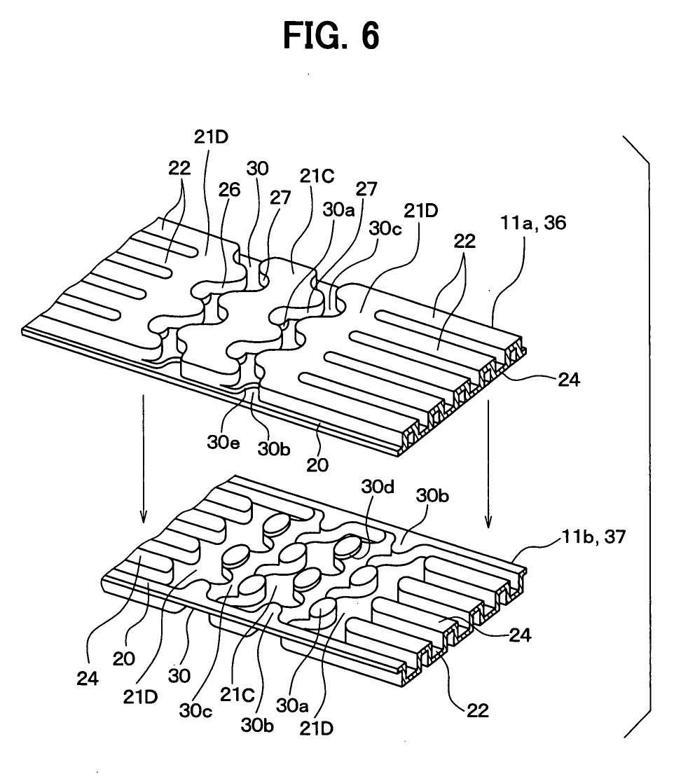

Further, as shown in FIG. 6, the tube 11 may be formed by connecting works 36, 37, after the board members 11a, 11b are separately formed by using the work 36, 37, respectively. Specifically, the shape pattern P of the board member 11a is repeatedly embossed to the work 36, and the shape pattern P of the board member 11b is repeatedly embossed to the work 37. Then, the work 36, 37 is cut such that a predetermined emboss part 21D of the shape pattern P is separated into the uniform cross-section areas 25 in the longitudinal direction. The works 36, 37 are set to face each other and to be in contact with each other. Then, the work 36 and the work 37 are connected by brazing so that the tube 11 can be produced.

When the works 36, 37 are set to face each other and to be in contact with each other, a part of the work 36 and a part of the work 37 are crimped in order to temporarily fix the works 36, 37 in the contact state. (The crimping process is not shown in the drawings.)

Next, operation of the heat exchanger 10 will be briefly described. High-temperature and high-pressure refrigerant discharged from the compressor (not shown) of the refrigeration cycle flows into the heat exchanger 10 through the connection block 14b, and is distributed into each tube 11 through the tank 14.

Refrigerant flowing through the tube 11 transmits heat to the tube 11 and the fin 12 connected to the tube 11. The transmitted heat is further transmitted to air flowing in a direction approximately perpendicular to the tube longitudinal direction, so that refrigerant is condensed into a liquid phase. The condensed refrigerant is collected in the tank 15 through each tube 11, and flows out of the heat exchanger 10 toward the expansion valve (not shown) through the connection block 15b.

Next, heat-exchange operation between refrigerant and air at the heat exchanging portion 13 of the heat exchanger 10 will be described. As shown of the arrow B in FIG. 4, when refrigerant flows through the tube 11, the refrigerant complicatedly meanders, so that a flow of the refrigerant can be agitated. Therefore, heat-transmitting performance can be improved, because a refrigerant side heat-transmitting rate can be improved.

In contrast, air flows outside of the tubes 11. When air flows in an area some distance from the tube 11, the air flows along the fin 12 as shown of the arrow C in FIG. 4. At this time, air absorbs heat from the fin 12 so as to cool the fin 12, and flows into a downstream side of the fin 12.

When air flows adjacent to the tube 11, the air absorbs heat from the tube 11 so as to cool the tube 11. Then, air flows into a downstream side of the tube 11. At this time, because the air flows and meanders through the air passage 30 as shown of the arrow D, a flow of the air can be agitated. Thus, heat-transmitting performance can be improved, because an air side heat-transmitting rate can be improved.

Further, because a contraction flow is generated when air flows into the air passage 30, the air side heat-transmitting rate can be improved. Further, because the air passage 30 increases heat-transmitting area of the tube 11, heat-transmitting amount from the tube 11 to air can be increased.

Further, due to the step parts 30d, 30e of the air passage 30, air flowing through the air passage 30 can be more agitated. Therefore, the air side heat-transmitting rate can be more improved.

Furthermore, because the insert hole 14a, 15a of the tank 14, 15 has a shape similar to the cross-section of the uniform cross-section portion 25 of the tube 11, a gap between the insert hole 14a, 15a and an outer face of the tube 11 can be reduced. Therefore, the tube 11 can be easily and accurately connected to the tank 14, 15.

Here, because end portions of the tube 11 in the tube longitudinal direction have approximately the same cross-section, the tube 11 can be easily inserted into the insert hole 14a, 15a of the tank 14, 15. Further, because the board members 11a, 11b are connected to each other through the joint 24, the uniform cross-section portion 25 of the tube 11 can be secured to have appropriate pressure-resistant strength.

Further, because the emboss parts 21C, 21D are formed in the same embossing process, the uniform cross-section portion 25 and the air passage 30 can be formed in the same process. Therefore, productivity for the tube 11 can be better.

Here, when the uniform cross-section portion 25 is formed only at the end portions of the tube 11, the uniform cross-section portion 25 has to be formed at a uniform interval. If a dimension of the tube 11 in the tube longitudinal direction is changed, the interval has to be changed. Thus, it is difficult and hard to correspond to many kinds of the dimensions of the tubes 11. When the uniform cross-section portion 25 and the air passage 30 are formed by the rollers 34, 35, the rollers 34, 35 have to correspond to a length of the tube 11. When the uniform cross-section portion 25 and the air passage 30 are formed by pressing, pressing-dies have to correspond to a length of the tube 11.

However, according to this embodiment, the uniform cross-section portion 25 is formed in the mid-part of the tube 11 in the tube longitudinal direction. Therefore, the plural lengths of tubes 11 can be formed without changing the rollers 34, 35 or the pressing-dies.

For example, when the tube 11 is cut on the chained-lines A in FIG. 3, a tube having a shorter length can be formed. Thus, the plural lengths of tubes 11 can be formed by common rollers 34, 35 or common pressing-dies. Therefore, the plural lengths of tubes 11 can be easily produced.

Here, the roller 34 (35) has the protrusions 34a, 34b (the recesses 35a, 35b) corresponding to the emboss parts 21C, 21D. If the uniform cross-section portion 25 and the air passage 30 are irregularly arranged in the tube longitudinal direction, the protrusions 34a, 34b and the recesses 35a, 35b corresponding to the emboss parts 21C, 21D have to be formed on the rollers 34, 35 in the irregular order, so that an external diameter of the roller 34, 35 may be increased.

However, according to this embodiment, the shape pattern P having the single emboss part 21C and the single emboss part 21D is repeatedly arranged in the tube longitudinal direction. Therefore, the rollers 34, 35 have only protrusions and recesses corresponding to the shape pattern P. Thus, the external diameter of the roller 34, 35 can be reduced, and the tube 11 can be easily produced.

The shape pattern P may be any suitable shape without departing from the scope of the present disclosure. The shape of the air passage 30 may be any suitable shape without departing from the scope of the present disclosure. For example, the air passage 30 may have a shape disclosed in JP-A-2004-3787.

Further, the tube 11 is constructed with the board members 11a, 11b in the above description. Alternatively, the tube 11 may be formed by extrusion. In this case, one flat face of the flat tube 11 is pressed toward the other flat face side, so that plural recesses recessed toward the other flat face side are formed in the one flat face. Due to the plural recesses, the joint 24 and the air passage 30 can be formed.

Further, the heat exchanger 10 is used in the condenser for condensing refrigerant. Alternatively, the heat exchanger 10 may be generally and widely used for exchanging heat between fluids.

Such changes and modifications are to be understood as being within the scope of the present invention as defined by the appended claims.

What is claimed is:

1. A tube for exchanging heat between inside fluid flowing in the tube in a tube longitudinal direction, and outside fluid flowing in an outside passage meanderingly extending in an outside fluid flowing direction outside of the tube, the tube comprising:
a uniform cross-section portion disposed at least at each end of the tube longitudinal direction, in which a cross-section is approximately uniform in the tube longitudinal direction, the uniform cross-section portion having
an inside passage constructed with a first inner face and a second inner face, which are spaced from each other, wherein the inside fluid flows through the inside passage, and
a joint constructed with the first inner face and the second inner face, which are connected to each other to increase a pressure-resistant strength, wherein
the joint and the inside passage are alternately arranged in the uniform cross-section portion in a direction approximately perpendicular to the tube longitudinal direction.

2. The tube according to claim 1, wherein
the uniform cross-section portion is further disposed at a mid-part of the tube longitudinal direction.

3. The tube according to claim 2, wherein
the uniform cross-section portion is arranged in a first area,
the outside passage is arranged in a second area, and
the first area and the second area are alternately arranged in the tube longitudinal direction.

4. The tube according to claim 1, wherein
the first inner face and the second inner face are constructed with opposing faces of two board members,
the inside passage is constructed with an emboss part protruding outward from a base of the board member, and
the joint is constructed with the base of the board member.

5. A method of producing the tube according to claim 1, the method comprising:
forming the outside passage and the uniform cross-section portion on a work having a band plate shape such that a longitudinal direction of the work corresponds to an inside fluid flowing direction;
contacting ends of the work in a narrow side direction to each other by bending the work at an approximately center part of the work in the narrow side direction; and
cutting the work at a forming part of the uniform cross-section portion.

6. A method of producing the tube according to claim 1, the method comprising:
forming the outside passage on a first work having a band plate shape, and the uniform cross-section portion on a second work having a band plate shape, such that each longitudinal direction of the first and second works corresponds to an inside fluid flowing direction;
cutting the first and second works at a forming part of the uniform cross-section portion; and
contacting the first and second works to each other to oppose to each other.

7. A heat exchanger including a plurality of the tubes according to claim 1, and a tank, wherein
the tank has an insert hole, through which the uniform cross-section portion is inserted to be connected, and
the insert hole has a shape corresponding to the cross-section of the uniform cross-section portion.

8. A tube for exchanging heat between a first fluid flowing inside of the tube, and a second fluid flowing outside of the tube, the tube comprising:
a uniform cross-section portion, in which a cross-section is approximately uniform in a longitudinal direction, the uniform cross-section portion having
an inside passage constructed with a first inner face and a second inner face, which are spaced from each other, wherein the first fluid flows through the inside passage, and
a joint constructed with the first inner face and the second inner face, which are connected to each other to increase a pressure-resistant strength, wherein the joint and the inside passage are alternately arranged in the uniform cross-section portion in a width direction approximately perpendicular to the longitudinal direction; and
an outside passage portion, in which an outside passage meanderingly extends in the width direction, and the second fluid flows through the outside passage, wherein
the uniform cross-section portion is disposed at least two ends of the outside passage portion in the longitudinal direction.
